(19) United States
(12) Patent Application Publication
(10) Pub. No.: US 2014/0058461 A1
(43) Pub. Date: Feb. 27, 2014

(54) Fenestrated Bone Screw

(71) Applicant: Michael Black, Swarthmore, PA (US)

(72) Inventor: Michael Black, Swarthmore, PA (US)

(21) Appl. No.: 13/595,181

(22) Filed: Aug. 27, 2012

(51) Int. Cl. A61B 17/86

(52) U.S. Cl. 606/314

(57) ABSTRACT

Improved bone screws are provided. The bone screws comprise a head portion that is connected to a shaft portion. The shaft portion can comprise one or more fenestrations that extend along a length of the screw. The fenestrations can be straight, or they can include angled bends and arcuate curves. At least a portion of the bone screws can be shot-peened or cold-worked in order to increase the strength of the bone screws to allow for use in, among other regions, the cervical region of the spine.

### FIELD OF THE INVENTION

The present invention is generally directed to orthopedic implants and in particular, osteo-inductive bone screws.

### BACKGROUND OF THE INVENTION

Numerous procedures exist to alleviate pain caused by bone disease, trauma and fracture. During surgery, a number of implants, such as plates and stabilization systems, are used during treatment. These systems rely on bone screws that secure the implants to bone. There is thus a need to provide improved bone screws.

### SUMMARY OF THE INVENTION

Various embodiments of orthopedic implants are provided. In some embodiments, a bone screw is provided comprising a head portion comprising one or more slits. The bone screw further comprises a shaft portion connected to the head portion, the shaft portion comprising a plurality of threads and a tapered distal end. The shaft portion further comprises a fenestration having a first section and a second section, wherein the first section extends along a first direction and the second section extends along a second direction.

In other embodiments, a bone screw is provided comprising a head portion comprising one or more slits. The bone screw further comprises a shaft portion connected to the head portion, wherein the shaft portion comprises a plurality of threads. The shaft portion further comprises a curved fenestration having at least one arc pattern along a length of the shaft portion.

In other embodiments, a bone screw is provided comprising a head portion. The bone screw further comprises a shaft portion connected to the head portion, wherein the shaft portion comprises a plurality of threads. The shaft portion further comprises a fenestration having a length that extends along a majority of the length of the bone screw.

### BRIEF DESCRIPTION OF THE DRAWINGS

The invention will be more readily understood with reference to the embodiments thereof illustrated in the attached figures, in which:

FIGS. 1A-1C illustrate an improved fenestrated screw having an angled fenestration according to some embodiments.

FIGS. 2A-2C illustrate an improved fenestrated screw having a curved fenestration according to some embodiments.

FIGS. 3A-3C illustrate an improved fenestrated screw having a straight, continuous fenestration according to some embodiments.

FIGS. 4A-4C illustrate an improved fenestrated screw having parallel fenestrations according to some embodiments.

### DETAILED DESCRIPTION OF THE ILLUSTRATED EMBODIMENTS

Embodiments of the invention will now be described. The following detailed description of the invention is not intended to be illustrative of all embodiments. In describing embodiments of the present invention, specific terminology is employed for the sake of clarity. However, the invention is not intended to be limited to the specific terminology so selected. It is to be understood that each specific element includes all technical equivalents that operate in a similar manner to accomplish a similar purpose.

The present application describes bone screws having improved mechanical properties. In particular, the bone screws described herein have unique fenestrations that can accommodate and guide different material (e.g., bone cement) through different portions of the bone screw, thereby increasing the effectiveness of the material.

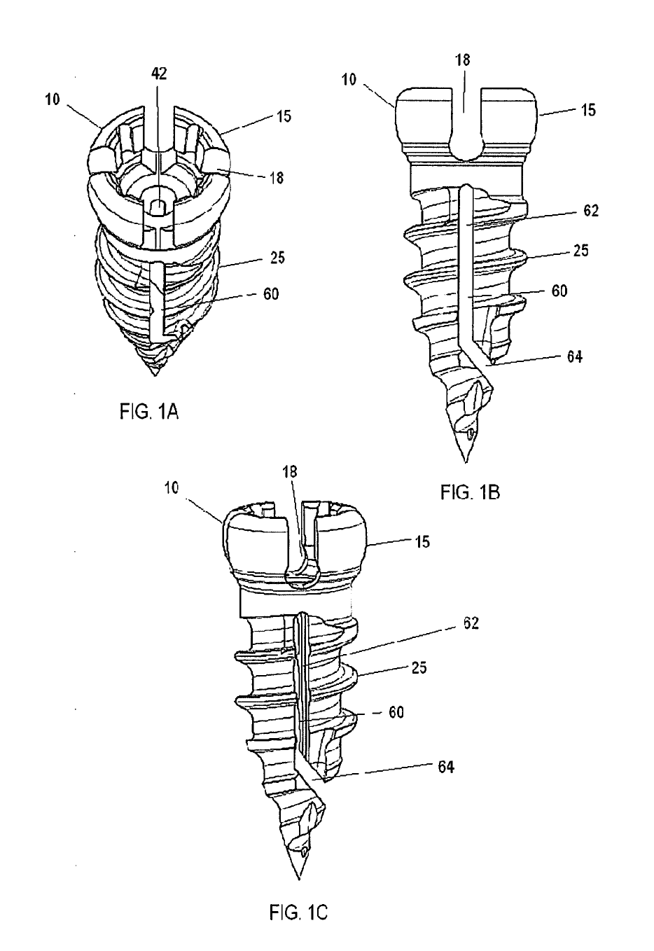

FIGS. 1A-1C illustrate an improved fenestrated screw having an angled fenestration according to some embodiments. The fenestrated screw 10 includes a head portion 15 connected to a threaded shaft portion 25. The threaded shaft portion 25 includes an angled fenestration 60 that extends along a longitudinal axis of the fenestrated screw 10, as discussed in more detail below.

The fenestrated screw 10 includes a head portion 15 having one or more openings or slits 18 formed therein. In some embodiments, the slits 18 are configured to allow for compressibility of the head portion 15. In addition, in other embodiments, the slits 18 are configured to accommodate one or more instruments, such as an insertion tool, for securely gripping the head portion 15 of the screw. As shown in FIG. 1A, the slits 18 are positioned symmetrically around the head portion 15 of the screw 10. However, in other embodiments, the slits 18 need not be positioned symmetrically around the head of the screw. As shown in FIG. 1A, the screw 10 includes four slits 18. However, in other embodiments, the screw can contain two, three, five or more slits.

The head portion 15 is operably connected to a threaded shaft portion 25. In some embodiments, the head portion 15 is formed monolithically with the shaft portion 25. In other embodiments, the head portion 15 is formed separately and then joined with the shaft portion 25. The shaft portion 25 is configured to have a tapered distal end that can be inserted into a vertebral body. In some embodiments, the threaded shaft portion 25 comprises a single thread, while in other embodiments, the threaded shaft portion 25 comprises a multi-diameter (e.g., dual-diameter) thread.

As shown in the top perspective view of FIG. 1A, the fenestrated screw 10 can be cannulated such that an inner opening 42 extends from the head portion 15 to the shaft portion 25 of the screw 10. In some embodiments, the inner opening 42 can be open and/or in fluid contact with the fenestration 60 (discussed in more detail below). Accordingly, material that is deposited through the inner opening 42, such as bone cement, can be advantageously distributed through the fenestration 60.

The fenestrated screw 10 includes a novel, angled fenestration 60. As shown in FIG. 1B, the fenestration 60 can extend along a longitudinal axis of the bone screw 10. In some embodiments, the fenestration 60 can extend along a majority of the length of the threaded shaft 25, thereby advantageously accommodating material along a majority of the length of the bone screw. For example, injected bone cement material, which can be used to secure the fenestrated screw 10 in bone, can now extend along a majority of the length of the bone screw 10, thereby improving the securing of the fenestrated screw in bone. In some embodiments, the fenestration 60 opens to the cannulated mid-portion of the fenestrated screw, such that material that is deposited through the cannulated portion can advantageously seep into the fenestration 60, and vice versa.

As shown best in FIGS. 1B and 1C, the fenestration 60 of the fenestrated screw 10 includes a first section 62 that extends along one direction and a second section 64 that extends along a different direction. In the illustrated embodiment, the first section 62 of the fenestration transitions into the second section 64 via an angled corner. As shown in the illustrated embodiments, the first section 62 comprises a straight portion that extends along a longitudinal length of the threaded shaft 25, which then transitions via an angled corner into the angled second section 64. In some embodiments, the angled corner of the fenestration 60 advantageously serves as a guide to direct material, such as fluids, along a certain direction along the length of the fenestration. In some embodiments, the first section 62 of the fenestration 60 can transition into the second section 64 via a sharp angle.

As shown in the side view of the screw 10 in FIG. 1B, the angled fenestration 60 can be formed such that the first section 62 of the fenestration 60 is formed in an interior portion of the screw 10 (e.g., along the middle longitudinal axis of the screw), while the second section 64 is formed to extend from an interior portion of the screw 10 to an outer edge of the screw 10. In some embodiments, the fenestration 60 effectively splits a portion of the threaded shaft 25 into two bodies. In other embodiments not shown, the fenestrated screw 10 can have an angled fenestration 60 that is formed completely in an interior portion of the screw 10 without extending to an outer edge of the screw 10. As the fenestration 60 can be made large and angled, this advantageously reduces the possibility of axial rotation and/or general screw toggling once cement or other biomaterial dries or fuses along the fenestration.

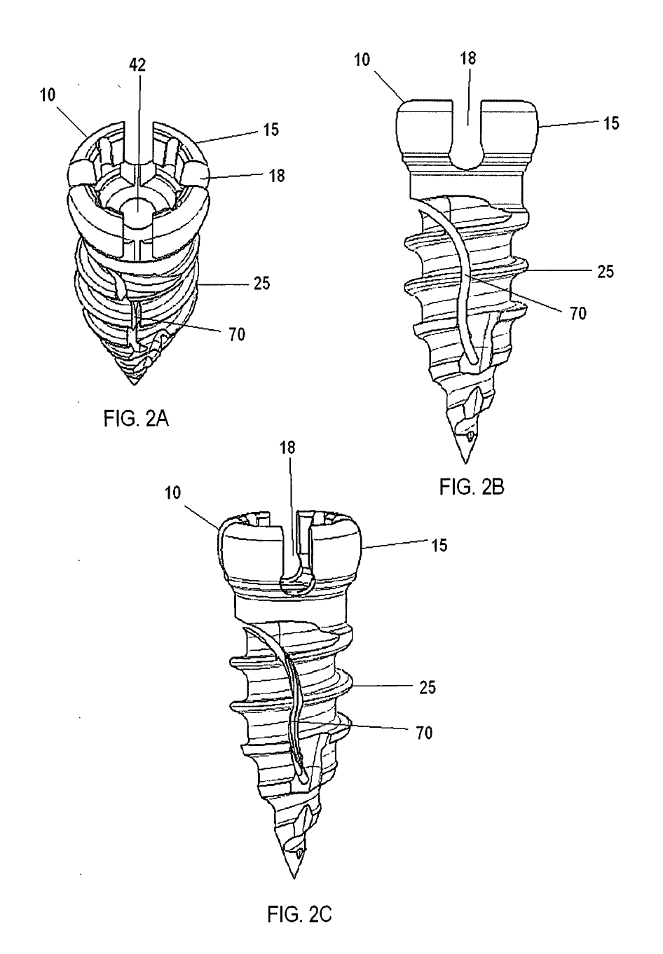

FIGS. 2A-2C illustrate an improved fenestrated screw having a curved fenestration according to some embodiments. Like the fenestrated screw discussed in FIGS. 1A-1C, the fenestrated screw 10 in FIGS. 2A-2C comprises a head portion 15, a shaft portion 25 and a fenestration therethrough. The fenestration 70, however, does not have an edge or corner, but rather is comprised of a continuous curve.

As shown in FIGS. 2B and 2C, the fenestration 70 comprises a continuously curved opening or window formed along a portion of the shaft portion 25. In some embodiments, the fenestration 70 extends along a majority of the length of the shaft portion 25, thereby allowing material to effectively seep along a majority of the length of the screw 10. As shown in FIG. 2B, the fenestration 70 advantageously extends from an interior portion of the screw 10 all the way to an outer edge of an exterior portion of the screw 10. In addition, as shown in the illustrated embodiments, the fenestration 70 comprises at least two arcuate portions. Such arcuate portions can advantageously provide a good distribution of material through the fenestrations, while properly maintaining sufficient strength in the fenestrated screw 10. However, one skilled in the art will appreciate that it is possible to have a fenestration 70 with a single arc or more than two arcs. Furthermore, in some embodiments, the fenestration 70 opens to and is in fluidic contact with the inner opening 42 of the screw 10. As the fenestration 70 can be made large and curved, this advantageously reduces the possibility of axial rotation and/or general screw toggling once cement or other biomaterial dries or fuses along the fenestration.

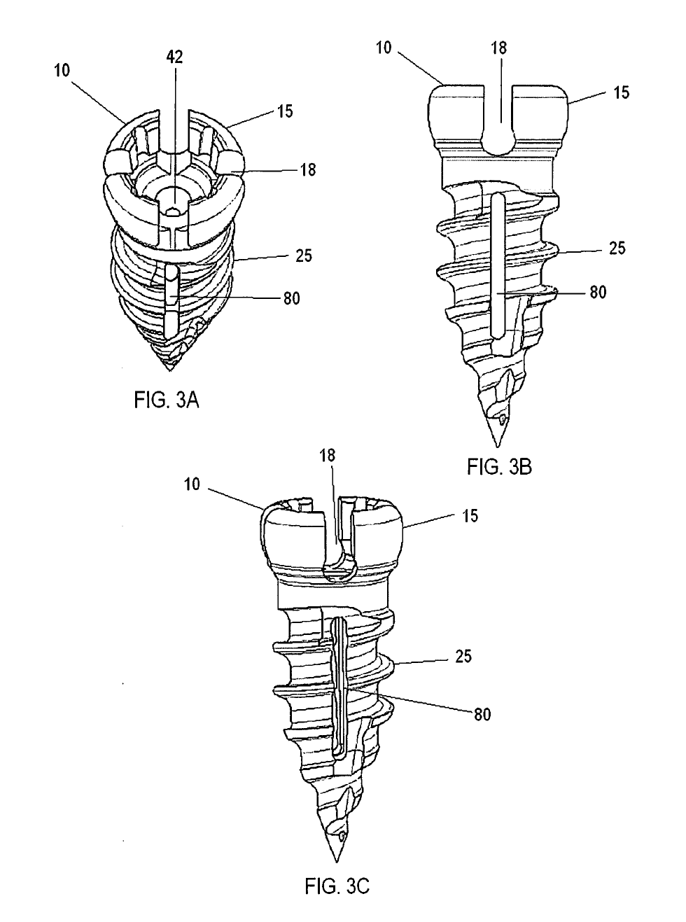

FIGS. 3A-3C illustrate an improved fenestrated screw having a straight, continuous fenestration according to some embodiments. Like the screws discussed above, fenestrated screw 10 includes a head 15 and a shaft 25. The screw 10 also includes a fenestration 80. However, the fenestration 80 comprises a substantially straight window or opening that is formed along a longitudinal length of the screw 10. While the fenestration 80 is substantially vertical and aligned along the mid-line of the screw, in other embodiments, the fenestration can be non-vertical (e.g., diagonal) and/or can be shifted away from the mid-line of the screw. In some embodiments, the fenestration 80 extends along a majority of the length of the shaft 25. Furthermore, in some embodiments, the fenestration 80 opens to and is in fluidic contact with the inner opening 42 of the screw 10.

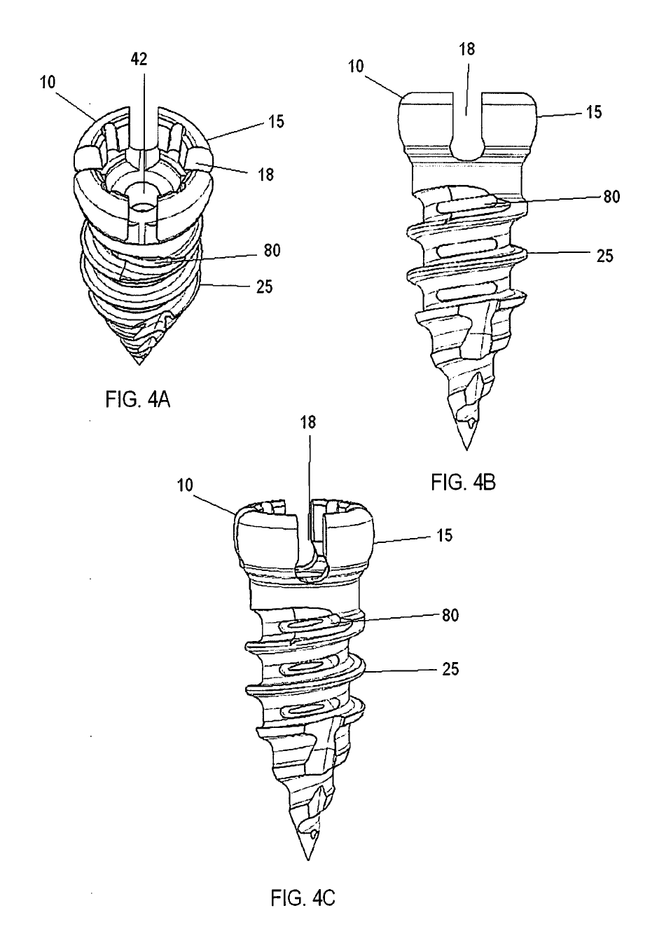

FIGS. 4A-4C illustrate an improved fenestrated screw having a plurality of angled, discontinuous fenestrations according to some embodiments. Like the screws discussed above, fenestrated screw 10 includes a head 15 and a shaft 25. However, the screw 10 includes a plurality of fenestrations 90. As shown in FIGS. 4B and 4C, the fenestrations 90 are slightly angled relative to a longitudinal mid-line of the screw 10. In addition, the fenestrations 90 are illustrated as substantially parallel to one another, although in other embodiments, the fenestrations 90 can be at non-parallel angles relative to one another. In the illustrated embodiment, there are three fenestrations 90; however, in other embodiments, there can be single, double, quadruple or more fenestrations. Advantageously, by having multiple fenestrations 90, the fenestrations 90 can distributed with ease around different portions of the screw, while maintaining the integrity of the strength of the screw. In some embodiments, one or more of the fenestrations 90 opens toward and is in fluidic contact with the inner hole 42 of the screw 10.

The fenestrated screws described above can be used in a variety of medical procedures, including surgery of the spine adjacent the lumbar, thoracic and even cervical vertebrae. While it has been difficult to include screws with fenestrations in cervical vertebrae, as such screws are small in size and may have reduced strength due to fenestrations, it has been found that performing a process on the screw material, such as shot-peening or cold-working, can enhance the fatigue life and postpone the development of surface cracks that may be found. Accordingly, in some embodiments, the material of the screws described above have been shot-peened or cold-worked in order to produce small, yet strong, screws for use in the cervical region of the vertebrae. In addition, by shot-peening or cold-working the material of the screws, this can increase the strength of the screws, thereby enhancing the pull out strength of a screw without negatively impacting the strength of the overall screw.

While the invention herein disclosed has been described by means of specific embodiments and applications thereof, numerous modifications and variations can be made thereto by those skilled in the art without departing from the scope of the invention.

What is claimed is:

1. A bone screw comprising:
a head portion comprising one or more slits; and
a shaft portion connected to the head portion, the shaft portion comprising a plurality of threads and a tapered distal end, the shaft portion further comprising a fenestration having a first section and a second section, wherein the first section extends along a first direction and the second section extends along a second direction.

2. The bone screw of claim 1, wherein the head portion comprises four symmetrical slits.

3. The bone screw of claim 1, wherein the shaft portion comprises dual-diameter threading.

4. The bone screw of claim 1, wherein the first section of the fenestration extends substantially along a midline of the bone screw.

5. The bone screw of claim 1, wherein the first section of the fenestration transitions into the second section of the fenestration via an angled corner.

6. The bone screw of claim 1, further comprising a cannulated opening that extends through the head portion and the shaft portion.

7. The bone screw of claim 6, wherein the cannulated opening is in fluid contact with the fenestration.

8. A bone screw comprising:
a head portion comprising one or more slits; and
a shaft portion connected to the head portion, the shaft portion comprising a plurality of threads, the shaft portion further comprising a curved fenestration having at least one arc pattern along a length of the shaft portion.

9. The bone screw of claim 8, wherein at least a portion of the bone screw is shot-peened.

10. The bone screw of claim 8, wherein at least a portion of the bone screw is cold-worked.

11. The bone screw of claim 8, wherein it is configured for use in a cervical region of a spine.

12. The bone screw of claim 8, wherein the curved fenestration includes at least two arc patterns formed along a length of the shaft portion.

13. The bone screw of claim 8, wherein the fenestration extends along a majority of the length of the screw.

14. The bone screw of claim 8, wherein the shaft portion comprises dual-diameter threading.

15. A bone screw comprising:
a head portion; and
a shaft portion connected to the head portion, the shaft portion comprising a plurality of threads, the shaft portion further comprising a fenestration having a length that extends along a majority of the length of the bone screw.

16. The bone screw of claim 15, wherein the fenestration has a first section that transitions into a second section via an angled corner.

17. The bone screw of claim 15, wherein the fenestration has a smooth, continuous curvature from a first end to a second end.

18. The bone screw of claim 15, wherein at least a portion of the bone screw is shot-peened and/or cold-worked.

19. The bone screw of claim 15, wherein the fenestration extends substantially along a longitudinal axis of the bone screw.

20. The bone screw of claim 15, wherein the shaft portion is tapered.
